(19) United States
(12) Patent Application Publication
(10) Pub. No.: US 2002/0150952 A1
(43) Pub. Date: Oct. 17, 2002

(54) Methods for measuring lymphocyte activation by mitogens and antigens

(76) Inventors: Peter Sottong, Arnold, MD (US); Richard J. Kowalski, Rosedale, MD (US)

Correspondence Address: Whitham, Curtis & Christofferson, PC, 11491 Sunset Hills Road, Suite 305, Reston, VA, 20190, US

(21) Appl. No.: 10/123,163

(22) Filed: Apr. 17, 2002

Related U.S. Application Data
(60) Provisional application No. 60/283,935, filed on Apr. 17, 2001.

(51) Int. Cl. G01N 33/567

(52) U.S. Cl. 435/7.21

(57) ABSTRACT

Methods for measuring the function of lymphocytes and their responses to mitogens or specific antigens, with or without co-stimulatory agents, is provided. The methods are suitable for measurement of the responses of lymphoid cells when they are a subpopulation of cells, and also for measuring the function of specific subsets of lymphoid cells, each subpopulation or subset of a subpopulation having characteristic determinants on their cell surface. The invention also relates to test kits used in performing such methods. The methods of the invention facilitate screening of complex biological fluids, such as whole blood, by means of incubating a sample of the fluid with a mitogen or antigen, with or without co-stimulatory elements, separating the selected subset of interest, e.g., via affinity separation, and detecting the presence of an internal cellular component, advantageously ATP, that is increased as a result of the response.

### BACKGROUND OF THE INVENTION

1. Field of the Invention

The invention relates to rapid methods for measuring the function of lymphocytes and their responses to mitogens or specific antigens. In particular, the methods rapidly detect the presence of an internal cellular component, advantageously ATP, that is increased as a result of an immune response.

2. Background of the Invention

The immune system is central to control of infectious diseases and cancer. Lymphocytes, a class of white blood cells, are critical cell types that are responsible for the activities of the immune system. Lymphocytes are divided into two major categories. T lymphocytes and B lymphocytes. Overall assessment of the function of the immune systems and, in particular, lymphocytes is important in assessment of immunodeficiency caused by: genetic factors, infectious disease such as (HIV), drugs following transplantation, stress, aging, or nutritional deprivation.

Lymphocytes express receptors on the cell surface that bind with specific antigens or epitopes. Exposure to the antigen results in expansion of the population of lymphocytes that is reactive to that antigen. Measurement of the response of the immune system to a specific antigen can be useful in diagnosis of infectious disease, hypersensitivity to certain agents, exposure to immunologically reactive drugs, or response to vaccination.

The function of B lymphocytes or their response to a specific antigen can be assessed by measuring the level of specific antibody in bodily fluids such as blood, saliva or urine. The function of T lymphocytes (T cells) or their response to specific antigens is more difficult to measure. Measurement of the functions of T cells is complicated by a number of factors. First, there are several different subsets of T cells with different functions. These subsets have been classified in part by the expression of characteristic cell surface markers and in part by a variety of functional assays including measurement of cytokines. Second, T cells respond to antigens only when they are presented by other cells in the context of major histocompatibility antigens on the surface of the presenting cell. Third, many of the functions of T cells depend on cell-cell contact with effector cells or the functions are fairly localized.

Current methods for measuring immune function are tedious, time consuming, and poorly adapted to the clinical laboratory setting. Methods that are currently used for direct or indirect measurement of immune function include: methods based on counting the number of lymphoid cells or different subsets; methods based on measuring the proliferation of lymphocytes, methods based on measurement of cytotoxic activity or secretion of cytokines, and methods used in vivo such as skin tests and adoptive transfer. These methods are described in detail in the literature (see for example Groeneveld et al, Journal of the International Federation of Clinical Chemistry, 6: 84-94; 1994; Clough and Roth. JAVMA 206:1208-1216. 1995).

The methods most commonly used in the clinical laboratories are based on counting the number of lymphoid cells or subsets. A variety of techniques have been described including immunofluorescence microscopy, immunocytochemistry, enzyme immunoassay, and flow cytometry. Flow cytometry, in particular, is widely used in clinical laboratory settings and is particularly useful in measurement of subsets of interest within a complex population of cells. For example, U.S. Pat. No. 4,727,020 to Recktenwald describes the use of two fluorescent channels to detect cells in a subpopulation specifically labeled with two different immunofluorescent agents. U.S. Pat. No. 4,284,412 to Hansen, et al. describes the use of fluorescence channels to detect forward and right angle light scattering by cells of different subpopulations in blood. Major disadvantages of flow cytometry include the requirement for complex and expensive equipment, the requirement for significant “hands on” time since each sample must be run using many manual steps, and the necessity of utilizing highly trained individuals to analyze results. These disadvantages are particularly acute in a clinical laboratory which must process multiple patient specimens daily and where the need for consistent and reliable results is extremely important.

U.S. Pat. No. 5,385,822 to Melnicoff et al. and U.S. Pat. No. 5,374,531 to Jensen disclose alternative methods to flow cytometry for enumerating the number of lymphocytes or a subset of lymphocytes within a mixed population of cells. The methods described in these patents involve coupling a detectable reporter substance to the biomembrane, or incorporating the reporter substance into the cell, then separating the subset or population of interest and detecting the reporter substance. These methods utilize affinity separation to isolate populations of interest which form a complex mixture of cells. While this technique offers improvements over flow cytometry, it is still based on cell counting techniques.

The major difficulty with all cell counting techniques is that they do not measure the function of specific cells or their responses to specific or non-specific stimuli, such as specific antigens or mitogens. Rather, these methods reflect the binding characteristics of the cell surface antigens. In addition, these methods are tedious and subject to poor reproducibility.

Direct measurement of responses of lymphocytes have included lymphoproliferation assays, cytotoxicity assays, and measurement of cytokines. In general, these methods require separation of white cells from the original sample followed by incubation with antigen or mitogen. Measurement of the function of specific subsets of lymphocytes requires extensive manipulations prior to the assay. For example, the requirement for antigen presenting cells means that additional cells have to be added back to the culture. Lymphoproliferation assays are based on the division of responding cells and are typically performed using radioactive isotopes. Because they evaluate the division of a small population of cells and require tissue culture, the assays take 3-10 days and are subject to significant variability based on the specific technique and the reagents used in the assay. Cytotoxic tests also require significant cell manipulation and are similarly highly variable depending on the specific conditions used. Cytokine assays can also be performed, but require many steps and separation of subsets of interest prior to stimulating the cells. U.S. Pat. No. 5,344,755 to McMichaels describes a modification of the cytotoxic assay based on initial immunomagnetic separation of T lymphocytes, but this method still requires extensive manipulation of effector cells. U.S. Pat. No. 5,344,755 provides an example of the use of cytokine measurements to assess immune status in HIV positive patients but is tedious and requires multiple steps. These methods require the separation of critical cell types, long incubation times, and in some cases, the use of radioactive substances. For these reasons, these methods have not been suitable for clinical applications.

Recently flow cytometry methods have been adapted to measure responses to antigen or mitogen stimulation through the expression of intracellular cytokines (e.g. U.S. Pat. Nos. 5,445,393; 5,656,446; 5,843,659). Since cytokines are normally released by cells in response to stimulation, flow cytometry-based methods require the addition of brefeldin to the culture to inhibit cytokine release, thus rendering the assays non-physiological. In addition, the cells require multiple incubations, centrifugation steps, and washing to stimulate, inhibit cytokine release, permeabilize cells, bind cell surface markers, fix, and then stain cells for visualization by the flow cytometer.

Affinity separation of cells using protein-coated magnetic particles or other types of solid supports such as polystyrene particles is known and is used as part of several of the methods cited above, see U.S. Pat. Nos. 5,374,531, 5,385,822, and 5,344,755. Various methods for sorting biological populations via affinity separations on solid supports have been described in the patent literature and elsewhere. See, for example, U.S. Pat. Nos. 3,970,518, 4,710,472, 4,677,067, 4,666,595, 4,230,685, 4,219,411, 4,157,323; see also, E. T. Menz, et al., Am. Biotech. Lab. (1986); J. S. Kemshead, et al., Molec. Cell. Biochem., 67:11-18 (1985); T. Leivestag, et al., Tissue Antigens, 28:46-52 (1986); and J. S. Berman et al., J. Immunol., 138:2100-03 (1987). In performing such methods, a binding molecule (e.g., monoclonal antibody) is typically conjugated to a solid support such as magnetic particles or plastic beads, and added to a test sample under conditions which cause binding to a characteristic determinant on the analyte of interest. The cells complexed with the solid support are then separated from the uncomplexed cells by exposure to a magnetic field or filtration or some other suitable method, depending on the nature of the solid support. The use of this technology to separate certain subpopulations of lymphocytes from bone marrow cells prior to transplantation and to eliminate post-transplantation graft vs. host disease, has also been reported. See A. Butturini et al., Prog. Bone Marrow Transpl. 4:13-22 (1987). Other reported uses of this technology include the separation of tumor cells (see: Kemshead et al., B. J. Cancer 54:771-78 (1986) and the separation of lymphocytes subpopulations for subsequent functional evaluation.

The effect of co-stimulation with immobilized anti-CD3 plus anti-CD28 on long-term proliferation of CD4 lymphocytes has been reported. See B. L. Levine, et al., J. Immunol. 159:5921-5930 (1997). In this instance the interaction between dendritic cells and T cells is mimical, as it provides both the primary stimulatory signal through the CD3 complex and a co-stimulatory signal through the CD28 receptor. (See Garlie, et al., J. Immunotherapy 22(4):336-345 (1999).

Several problems arise when lymphocytes are separated by magnetic or other solid phase affinity techniques and then used for functional assays. For example, the interaction of a lymphocyte with the binding molecule can itself induce functional changes in the lymphocyte that may obscure later changes that are to be measured. In addition, the accessory cells required for response of the T cells may no longer be present especially if a specific subset of cells are isolated. Further, isolated cells are removed from the native environment and it is difficult to maintain the sterility of the sample required for further tissue culture.

U.S. Pat. No. 5,773,232 to Weir (the entire contents of which is hereby incorporated by reference) discloses methodolgy that addresses some of these drawbacks. Weir describes the discovery that activated T-cells display elevated levels of ATP, and that the level of activation of a population of T-cells can be ascertained by measuring the ATP levels. However, the method of Weir contemplates analysis for a 24-hour period.

It would be highly beneficial to have available a method to rapidly and sensitively detect activation of T cells that does not suffer from these drawbacks.

### SUMMARY OF THE INVENTION

The subject invention provides a convenient, reliable, and rapid method for analyzing the function of various sets or subsets of lymphocytes. The method of the invention involves exposing a population of cells to at least one inducing agent (e.g. a mitogen or an antigen, with or without a co-stimulatory agent); separating a desired subset of cells by means of the interaction of a specific binding substance that is attached to a solid phase with a cell surface determinant that is present on the cell subset of interest; lysing the separated cells; and measuring an intracellular component that is increased if the cells have responded to the inducing agent(s). All of these steps take place in less than 24 hours, and preferably in less than 3 or 4 hours, and most preferably in less than 1 hour.

In an advantageous embodiment of the invention, the functional activity of a set or subset of lymphocytes which is distinguished by a characteristic cell surface determinant and which is contained within a mixed cell population and which is measured by: exposing the sample to a mitogen or antigen, with or without a stimulatory agent; incubating the sample for a period of time; binding the set or subset of lymphocytes to a solid support through the interaction of the cell surface determinant and a specific binding substance which is linked to the solid support; washing the cells to remove any unbound cells as well as potentially interfering materials in the media; lysing the cells; and detecting the ATP in the solution. The results obtained can be compared against a known standard. Alternatively, the sample can be divided into two or more parts with at least one of the parts being incubated without addition of any inducing agent, while the second part is incubated with addition of at least one inducing agent.

One aspect of the invention is the determination of the response of lymphocytes to a mitogen. In this case, the response of a set of lymphocytes to a mitogen is a general measure of immune function or innate immunity. This application is of particular significance in the measurement of the effects of immunosuppressive drugs or agents, or viral induced immunosuppressive states. In this case, the responsiveness of an entire set of lymphocytes such as the T lymphocytes can be determined. Alternatively, the effect of a virus such as HIV can be assessed by evaluating the response to mitogens of a subset of T lymphocytes that express the CD4 cell surface determinant.

Another aspect of the invention is the determination of the response of lymphocytes to antigens that include but are not limited to infectious agents, drugs, chemicals, autoantigens, alloantigens or tumor antigens. This aspect of the invention is particularly important in monitoring the exposure of an individual to an infectious disease or agent, or to the diagnosis of hypersensitivity, autoimmune disease, organ transplant tolerance or rejection, or cancer. This aspect of the invention is also useful in monitoring vaccine efficacy and in assessment of immunotoxicity of chemicals, drugs, and industrial compounds.

Another aspect of the invention is that the response of T lymphocytes from various subsets including functional or differentiation based subsets to at least one inducing agent can be assessed based on the expression of different determinants specific for a functional, differentiation, or activation marker on the cell surface. In this aspect of the invention, at least one inducing agent is added to a sample and incubated for a period of time. Following the incubation, the cell subset of interest is isolated by binding the cells to a solid support through a determinant on the cell surface and a specific binding substance immobilized on the support.

The cells are washed and lysed, and the level of an intracellular component that increases as a result of the activation is measured.

The method of the invention is highly sensitive due to the measurement of an intracellular component whose level increases rapidly following lymphocyte exposure to an inducing agent, for example ATP, other metabolic intermediates such as NADP, or proteins involved in cell cycle regulation such as PCNA. In an advantageous embodiment, the level of ATP is measured utilizing the bioluminescent reaction of luciferin:luciferase. Bioluminescent measurement of ATP is a highly sensitive measure.

A further aspect of the invention is that the total time required for the method is generally less than 24 hours, and preferably 3 hours or less, and most preferably 1 hour or less (e.g. 30 minutes). The relatively short time period is an advantage in comparison to current methods which require 3-10 days to complete, or the method described in U.S. Pat. No. 5,773,232 to Weir which requires 24 hours to complete.

In another advantageous embodiment of this invention, the functional activity of a set or subset of lymphocytes contained within a mixed cell population, is measured by: exposing the sample to a mitogen or antigen, and at the same time to a solid support containing immobilized specific binding substance in order to bind a set of lymphocytes to the solid support through the interaction of the cell surface determinant and the specific binding substance which is linked to the solid support, washing the cells to remove any unbound cells as well as potentially interfering substance in the media, lysing the cells, and detecting ATP in the solution. These results can be compared against a known standard, or alternatively, the sample can be divided into two or more parts with at least one of the parts being incubated with a solid support containing immobilized specific binding substance and another being incubated with a solid support which does not contain immobilized specific binding substance. In a preferred embodiment of the invention, the immobilized specific binding substance is a co-stimulatory agent such as an antibody.

In another advantageous embodiment of this invention antigen-specific subsets of cells are selected by way of a magnetic particles coated with specific peptides, recombinant molecules, or more complex antigens (e.g. whole virus or bacteria, or lysates, etc.). Removal of these antigen-specific cells from other cell populations prior to lysing and measurement of the ATP content, overcomes low precursor frequency by allowing detection in the absence of non-specific signal.

In one embodiment of the invention, test kits are provided for performing the methods of the invention. Test kits typically contain inducing agents, the solid state with appropriate binding substance, the reagents required for detection of ATP levels, appropriate diluent and wash solutions, standards or instructions for preparing the same, and optionally, other accessories such as test tubes, magnetic separators, washers, and transfer pipettes, which are useful in carrying out the methods of the invention.

### BRIEF DESCRIPTION OF THE DRAWINGS

FIG. 1 shows the response of a blood sample to stimulation by a mitogen (phytohemaglutinin) and an antigen (tetanus toxoid). This time course shows the results of Example 1, and demonstrates measurable response as early as 30 minutes after stimulation. NS=no stimulation control; PHA=phytohemaglutinin, 1%; tetanus=tetanus toxoid, 1 ug/mL.

FIG. 2 shows the whole blood sample response before and after stimulation with phytomemaglutinin. This time course of response over 2-24 hours shows the results of Example 2. NS=no stimulation control; PHA=phytohemaglutinin, 1%.

FIG. 3 shows assay results of Example 3, in which responses to tetanus toxoid were measured 30 minutes to 3 hours after costimulation with immobilized CD3/CD28 antibody. ♦=NS (no stimulation) control; ▪=1 μg/mL tetanus toxin.

FIG. 4 shows the assay results described in Example 4, the T lymphocyte response distributions of apparently healthy adults (♦) and HIV infected donors (▪).

### DETAILED DESCRIPTION OF THE PREFERRED EMBODIMENTS OF THE INVENTION

The present invention provides sensitive and efficient methods for measuring the activation of a set or subset of lymphocytes (e.g., T cells or B cells) distinguished by some characteristic determinant expressed on the cell surface within a mixed cell population. In particular, the invention provides a method for measuring the response of lymphocytes to inducing agents which include mitogens, antigens, and co-stimulants, or combinations thereof.

The methods of the present invention involve measuring an intracellular component of the set or subset of cells which increases upon activation of the cells. In a preferred embodiment of the invention, the intracellular component is ATP. It is well known that ATP levels are indicative of metabolic activity. See, for example, Kangas et al, Med. Biol. 61:338-43 (1984) and Lundin et al. Meth. Enzymol. 133:27-42. Measurement of ATP levels have been used in studies of chemotherapeutic drugs and other agents of cell lines and have been used to monitor increases in biomass and cell number. ATP levels can be measured very sensitively using the bioluminescent reaction of firefly luciferase with luciferin. See, for example, Leach and Webster, Meth. Enzymol. 133:51-70. (1986). A number of methods for assessing ATP levels in bacteria or in somatic cells have been reported. See, for example, U.S. Pat. Nos. 3,690,832, 5,283,179, 4,144,134, 4,283,490, 4,303,752 each of which is hereby incorporated by reference.

The metabolic activity of the cell that responds increases significantly and this increase is reflected in significant increases in the level of ATP levels. Further, small changes in ATP levels or changes in ATP levels in a small number of cells can be measured due to the sensitivity of the luciferin:luciferase system. See Buttgereit, et al., Review Immunology Today, 192(21):194-199. (2000).

The invention addresses a major existing need for improved methods for detecting T cell activation. The present methods exhibit sensitivity comparable to or greater than methods heretofore available. These methods also permit the user to analyze multiple samples in a relatively brief time, eliminate the need for expensive equipment and highly skilled personnel to perform the method, and do not use radioactive materials.

Antigenic T cell activation responses are typically weaker than those provided by mitogenic stimulation. In addition, immunosuppressed or immunodeficient patients often display reduced T cell responses to stimuli. Therefore, methods that can enhance the sensitivity to antigenic stimulation can provide additional useful information for diagnostic or immune assessment purposes. Currently, tests which rely on clonal cell expansion as part of the assay methodology provide a means for detecting weak antigenic responses. However, because of the lengthy assay period required for proliferation (3-10 days) they are impractical for most clinical applications. The present invention provides methods which incorporate co-stimulatory agents of T lymphocytes in order to assess the early stages of T cell activation. The method does not require cell division in order to detect a significant level of intracellular component. Thus, the response can be measured after a relatively short incubation or exposure time frame (e.g. from about 0.5 to 24 hours), offering the desired increase in sensitivity while eliminating the long waiting period for results. In a preferred embodiment of the invention, the co-stimulatory agents are antibodies (e.g. anti-CD3 and anti-CD28) . In some embodiments, the antibodies are immobilized.

The methods of the invention can be used as an adjunct to or a replacement for methods and tests performed in the clinical laboratories to count the number of lymphocytes in different subpopulations. The methods described herein incorporate the required sensitivity of assays that take much longer time with the shortened time frame of cell counting assays. The utilization of standards and the provision of test kits incorporating all the necessary reagents for the practice of the present invention insures the reproducibility and consistency which is required for tests used in the clinical laboratory.

The methods of the invention have several advantages over current methods. In particular, the time required for response is significantly less than for other assays. The provision of all the materials required for the assay in a test kit and the simplicity of the measurement results in consistency in results from lab to lab. Further, multiple samples can be run simultaneously. The methods are simple, rapid, sensitive, and applicable to the clinical laboratory setting.

### Inducing Agents

In the context of this invention, inducing agents are substances that interact with lymphocytes such that the results of the interaction is a change in the state of the cell. In particular, “inducing agents” refer to substances that cause resting lymphocytes to become activated and that can also induce functional activity in the cell. In general, inducing agents fall into three classes: (i) mitogens that interact with all the lymphocytes of a particular subset and induce activation followed by proliferation in the responding cells; (ii) antigens that interact through specific receptors on limited subpopulations of cells; and (iii) co-stimulatory regulatory agents that interact through one or more receptors or binding proteins of many subpopulations of cells.

Mitogens for different populations of lymphocytes are known and include lectins, growth factors and lymphokines, phorbol esters, and other biochemical substances that are known to those versed in the art. Advantageous mitogens include phytohemagglutinin (PHA) and Concanavalin A (Con A).

Antigens react with a smaller subset of lymphocytes through specific receptors on the cell surface. Each lymphocyte has on its cell surface a receptor for a specific antigen or molecule. For B lymphocytes, the cell surface receptor is antibody that is membrane bound. For T lymphocytes, the cell surface receptor is the T cell receptor with recognized antigen that is presented in the context of major histocompatibility molecules on the surface of another cell. The response of the immune system to specific foreign invaders is based on the recognition of antigens by the receptors on the cell surface of these cells and the resultant functional activation that occurs as a result of this interaction. In general, the antigens that are bound to these cell surface receptors are small parts of larger molecules and can include parts of infectious agents, such as viruses, bacteria, fungi, and the like, as well as drugs, organic chemicals, and inorganic chemicals such as silicone, metals such as beryllium, and proteins such as tumor cell proteins, or proteins derived from implanted or transplanted organs. Advantageous antigens include gp120 protein (or peptide fragments thereof) of HIV virus envelope glycoprotein; outer surface proteins from bacteria such as Outer Surface Protein A, B, or C from Borrelia burgdorferi; SiO₂; disrupted inactivated Q fever cells; purified protein derivative (PPD); tetanus toxoid; or staphylococcus superantigen that binds to all T-cell receptors.

Co-stimulatory or regulatory agents act by associating with receptors either on or within the cell or by interacting with a variety of cellular components. For lymphocytes, molecules such as cytokines and receptor ligands bind to specific receptors on subsets of cells and can induce an activation or inhibition response. Small molecules such as steroids, drugs and metal ions can permeate the cell membrane and bind to factors that control cellular function. Many of these agents, including cyclosporin and tacrolimus (FK506), are immunosuppressive and inhibit lymphocyte responses. Other agents, including mycophenolic acid, rapamycin, calcium activated potassium channel blockers (clotrimazole, charybdotoxin, and nitrendipine) and nucleotide analogs (AZT), affect cellular metabolism by interactions with mitochondria, signal transduction pathways and biochemical pathways. Advantageous co-stimulatory agents that enhance lymphocyte responses include interleukins (IL-2, IL6, IL10, IL12, IL-1a), anti-CD3 antibodies, CD28-ligand or anti-CD28 antibodies, and CD49d-ligand or anti-CD49d antibodies. These co-stimulatory molecules regulate lymphocyte responses by magnifying the interactions between specific T-cell receptors and specific antigens or mitogens through increased receptor-ligand binding, secondary messenger pathways (inositol triphosphate, calcium release channels), and biochemical signaling (e.g. phosphorylation).

### Target Cells

The cell subsets of interest are present in test samples or specimens of varying origin including biological fluids such as whole blood, urine, stool, saliva, cerebrospinal fluid, amniotic fluids, tissue extracts, lavage fluids, tumor biopsies, transplant biopsies or they can be from culture. Cells of interest are of human or animal origin. Samples also include specimens from various biological origins that have been partially purified by density gradient centrifugation or other separation methods that are used to isolate partially purified samples of cells.

In analyzing a sample containing a cell subset of interest according to the method of the invention, the cell population suspended in its natural biological fluid or in a suitable biological or synthetic medium, is initially exposed to the mitogen or specific antigen with or without the addition of a stimulatory agent. In other words, the cells may be exposed to a mitogen, to an antigen, to a mitogen and a stimulatory agent, or to an antigen and a stimulatory agent. The use of antigens or mitogens in combination with stimulatory agents can be determined by measuring the ATP signal which best optimizes the desired response.

Those of skill in the art will recognize that the concentration of inducing agent required in order to elicit a measurable reaction in the cells of interest will vary from inducing agent to inducing agent. The optimal concentration of such an agent can be determined by titrating the agent and measuring the ATP signal which best optimizes the desired response.

It is a particular aspect of the invention that the exposure occurs for a relatively short period of time. Depending on the nature of the inducing agent or combination of inducing agents being examined, the time period of exposure may be about 2-30 minutes to about 72 hours or longer. In a preferred embodiment of the invention, the period of exposure is about from 4 hours to about 24 hours.

Of particular interest in diagnostic, therapeutic, and research purposes are measurement of the responses of T lymphocytes and lymphocyte subsets, including major functional subsets such as T helper cells and suppressor/cytotoxic cells to mitogens or to specific antigens. Quantitation of the responsiveness of a specific subset of T lymphocytes is important in certain physiological conditions. For example, individuals infected with human immunodeficiency virus lose responsiveness in the CD4 cell population to both mitogens and antigens prior to loss of activity in other cell subsets. Likewise, responsiveness of subclasses of T cells to mitogens is important in monitoring individuals that are potentially immunosuppressed due to chronic stress, chemotherapy, or drug treatment. Quantitation of the responsiveness of a subset of lymphocytes to specific antigens is important in measuring the individual's exposure to an infectious agent or to a drug or compound, or to determine a hypersensitivity reaction to a drug, chemical or food. In addition to the major functional subsets other cell subsets of interest include cells at different stages of differentiation and cells at different time points after initial interaction with an inducing agent or combination of inducing agents.

### Characteristic Determinants

The subsets of interest are distinguished by expression of characteristic cell surface determinants. In addition, cells from the same subset but at different stages of differentiation are distinguished by expression of characteristic determinants on the cell surface. Cells with different functional activity or at different times after the initial interaction with an inducing agent may also express different cell surface determinants.

In this invention, the term “characteristic determinant” denotes an element that identifies or determines the nature of something. When used in reference to the methods of the invention, “determinant” means a molecule expressed on the cell surface that characterizes the cell in some fashion. Cell-associated determinants include, for example, components of the cell membrane, (such as membrane bound proteins, glycoproteins, lipids or glycolipids, including cell surface antigens of either host cell or viral origin), histocompatibility antigens, and membrane receptors. Particular characteristic determinants within the scope of this invention include CD69, CD25, CD26, CD27, CD28, CD49d, CD71, and MHC Class I and II antigens.

A determinant is the portion of the cell that interacts with a specific binding substance. Cells are separated by means of the specific interactions between determinants on the cell surface and specific binding substances that are attached to solid phases. This process is referred to herein as “affinity separation.” Specific binding substances that may interact with cell surface determinants include antibodies capable of recognizing the determinants and synthetic complexes of antigen and cognate Class I or Class II receptor ligands (e.g. MHC Class I Tetramers and MHC Class II dimers).

### Binding Substances

Determination of the presence or quantity of cell subsets according to the methods of the invention is accomplished by the selective interaction between cells of the subset of interest and a specific binding substance. The specific binding substance used in the practice of this invention must exhibit selective recognition for the characteristic cellular determinant. In analyzing a mixed cell population for a subpopulation and/or subset having a characteristic cell surface antigen, for example, the specific binding substance can be the complementary antibody that immunospecifically recognizes the antigen of interest. Based on such selective recognition, the specific binding substance is capable of selective interaction and binding with the subset of interest to form complexes or aggregates which are physically or chemically separate from the test medium and other components therein which are not of interest. In one advantageous embodiment, blood specimens containing T lymphocytes and monocytes bearing the surface antigen CD4 are exposed to a specific binding substance comprising a CD4 monoclonal antibody.

The term “antibodies” as used herein includes monoclonal or polyclonal inimunoglobulins and immunoreactive immunoglobulin fragments. Other types of specific binding substances include lectins, hormones, cytokines, receptor ligands, etc. Monoclonal antibodies to particular cell surface determinants are of particular importance in this embodiment of the invention. For example, lymphocytes, which comprise a subpopulation of whole blood, can be selected by a monoclonal antibody which is directed against a leukocyte surface antigen. The CD45 antigen is uniformly expressed on all lymphocytes; however, the CD45 antigen is also expressed on monocytes. Therefore, if selective binding of lymphocytes is desired, it is necessary to select a CD45 monoclonal antibody which binds to significantly more binding sites per cell on lymphocytes than monocytes or that binds with higher strength to lymphocytes than to monocytes.

In a particularly advantageous embodiment, it is desirable to separate only T helper lymphocytes within a sample of whole blood. This is accomplished as described above, using monoclonal antibodies directed against the T cell surface antigens, such as CD4, or is accomplished using a combination of antibodies that react primarily with T helper lymphocytes. In another embodiment of the invention, lymphocytes that have been activated by exposure to specific antigen are separated by utilizing antibodies directed against antigens that are expressed only following activation on the cell surface. These antibodies react with one or a combination of the following cell surface antigens, CD25, CD69, CD71, CD45RO, CD45RA or MHC Class II antigens. Using these antigens for separation results in a significant amplification of the signal from the assay since only the cells that express these markers have responded to the signal from antigen or mitogen.

In another embodiment of the invention, the specific binding substance is an immobilized co-stimulatory agent, and the lymphocytes are incubated with the mitogen or antigen, plus the immobilized co-stimulatory agent/specific binding substance. For example, in one embodiment of the invention, lymphocytes are exposed to antigen and immobilized antibodies such as anti-CD3, anti-CD28, or both together, at the same time. This simultaneous costimulation results in an enhancement of the specific antigenic signal.

Specific binding substances are conveniently affixed to a solid phase or insoluble fluid phase to facilitate separation from the test medium. A variety of solid support materials can be used e.g., polystyrene, nylon or agarose beads, and are well known to those skilled in the art. In a particularly advantageous embodiment of the invention, the specific binding substance is affixed to a plurality of magnetic beads, which comprise ferromagnetic, paramagnetic or diamagnetic material. Techniques for attaching the specific binding substance to the beads are known to those skilled in the art. Suitable techniques include cross-linking, covalent binding, or physical absorption. Alternatively, a non-solid phase, primary specific binding substance is used in conjunction with a second or auxiliary specific binding substance which is capable of interacting selectively with the primary specific binding substance, and which is affixed to a solid phase. Representative primary and auxiliary specific binding substances useful for this purpose are: soluble murine antibody/Protein A affixed to a solid phase; soluble murine antibody/anti-mouse immunoglobulin raised in another species and affixed to a solid phase; biotinylated antibody/avidin affixed to a solid phase.

In the case where the sample being tested is derived from culture or is separated by density gradient separation prior to separation on a solid support, a simple separation procedure is sufficient to separate the subset of lymphocytes of interest from the rest of the cell population. In the case of more complex samples such as whole blood it is necessary at times to wash the complex to remove cells that are trapped or bound nonspecifically. A variety of solutions (e.g., 0.15M ammonium chloride, 1.0M potassium carbonate, 0.1M EDTA, pH 7.2) that specifically lyse red blood cells, platelets, or other potential contaminants are known to those versed in the art. In addition, solutions that contain other substances such as proteins, sugars, or salts or that are of specific pH values can be useful in reducing the nonspecific binding of other cell types or in eliminating or lysing cell types that are separate from those of interest (e.g., solution of Hank's buffered saline containing 10% of FCS is particularly useful). Upon separation and following washing, if necessary, the media is removed from the complex.

### Lysis

Following separation of the cells of interest, the separated cell population is lysed by the addition of a solution containing substances that can lyse lymphocytes. A variety of such solutions exist and are well known to those in the art. These solutions include distilled water, solutions containing detergents such as Triton-X or NP-40, and buffered solution such as HEPES containing 0.1M benzalkonium chloride, pH 7.4. It is important that the material and the solution chosen do not interfere with the system for measuring ATP, do not contain ATP, and do not degrade ATP.

It is a significant feature of the invention that the time from the exposure of the sample until the time that the cells are lysed is minimal, usually less than 2 hours preferably less than 1 hour. This is significant because interaction of lymphocytes with many antibodies against cell surface antigens can result in a response. This has been a significant problem in determining the function of specific cell types as the isolation of the cell type can induce cell activation.

### Measurement of ATP Levels

Following lysis of the cells, the level of ATP in the solution is measured. In an advantageous embodiment, the ATP is measured by the addition of a solution containing firefly luciferase and luciferin in the presence of magnesium ions. ATP can also be measured by other means including immunochemical or biochemical reaction systems.

The measurement of an intrinsic component of the cell is important because it eliminates an additional step as well as the intrinsic variability of any labeling process. Increased ATP has been used as a marker of increased cell mass, but has been relatively insensitive because all the cells in the population exhibit a baseline level of ATP. The subject invention works because the measurement of ATP is made following separation of the cell population of interest.

### Kits

According to another aspect of the invention, the different reagents, together with the various accessories used in practicing the methods of the invention, including media for dilutions, solid supports for immobilizing cells, lysis reagents, inducing agents, and wash buffers, one or more standards, or instructions for the preparation thereof are conveniently packaged in a test kit. The reagents included in the test kit may take various forms and are packaged dry together with appropriate diluents or may be supplied in ready to use form. In accordance with the methods of the present invention, kits for evaluating the T cells responses are envisioned. In particular, the invention includes a kit containing antigens or mitogens either in liquid or lyophilized form, paramagnetic beads coupled with an antibody for isolation of the predetermined subset of cells, cell culture media for dilution of samples, wash buffer for washing complexes, and associated reagents for performing the assay.

The following examples are provided to describe the invention in further detail. These examples are intended to illustrate specific applications of the methods of the invention and should in no way be construed as limiting the invention.

### EXAMPLES

### Example 1

### Measurement of the Response of T-Lymphocytes to Phytohemagglutinin (PHA) and Tetanus Toxoid Using Whole Blood as the Sample

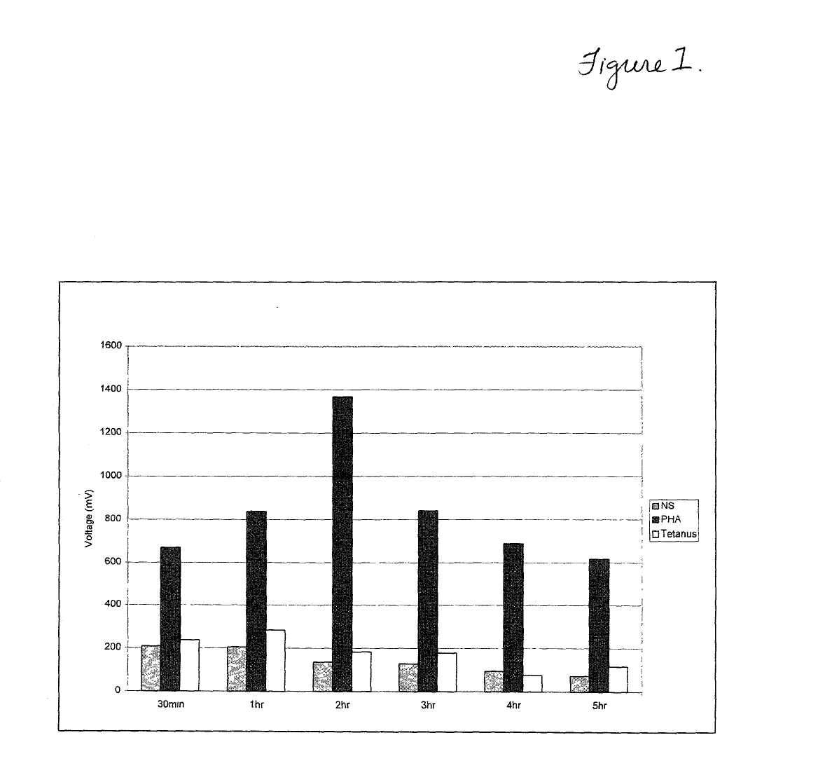

A blood sample was obtained from a normal donor and collected in heparin as an anticoagulant. Aliquots of the blood were diluted 1:10 in RPMI 1640 cell culture media (Biowhittaker, Walkersville, Md.). Replicates received either no addition, the mitogen PHA (Sigma-Aldrich, St. Louis, Mo.) at 1%, or the antigen Tetanus Toxoid (University of Massachusetts Medical Center, Jamaica Plains, Mass.) at 1 ug/mL final concentration. Samples were incubated at 37° C. At various time points (30 min, 1 hr, 2 hr, 3 hr, 4 hr and 5 hr), 100 uL of the samples were removed, incubated with paramagnetic beads coated with the binding substance anti-CD4 (Dynal, Oslo, Norway) (20 uL) for 30 minutes at room temperature. The cells and beads were placed next to a permanent magnet that was positioned such that the magnet field was in a direction perpendicular to gravity. Bead:cell complexes were washed (3 times in RPMI 1640) and lysed in a detergent solution. Assay Reagent (luciferin/luciferase) was added and the amount of ATP in the samples was quantified using a luminometer. The amount of ATP in stimulated samples was compared to that in unstimulated samples to determine the level of activation. FIG. 1 shows the results of this assay.

The results showed a statistically significant increase in ATP over background in response to PHA after 30 minutes. The response continues through 5 hours. An increase in ATP over background in response to Tetanus Toxoid is significant after 5 hours of incubation.

This example demonstrates that the method of the present invention successfully detects the activation of a subset of T lymphocytes in response to a mitogen within 30 minutes. Detection of the activation of the T lymphocyte subset by an antigen was effected within 5 hours.

### Example 2

### Whole Blood Response After Stimulation With Phytohemmaglutinin (PHA)

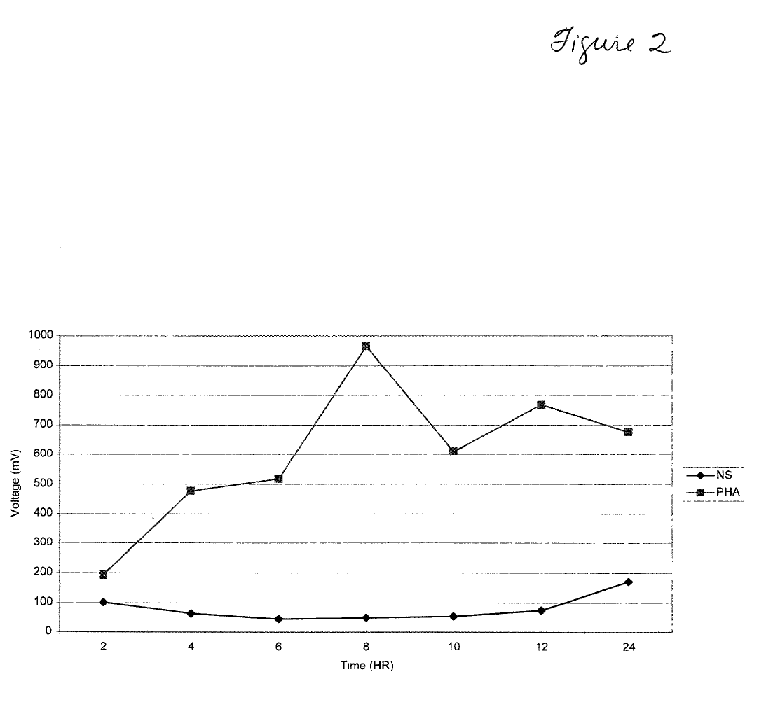

A blood sample was obtained from a normal donor and collected in heparin as an anticoagulant. Aliquots of the blood were diluted 1:10 in RPMI 1640. Replicates received either no addition or the mitogen PHA at 1% final concentration. Samples were incubated at 37° C. At various time points (2 hr, 4 hr, 6 hr, 8 hr and 24 hr), 100 uL of the samples were removed, incubated with anti-CD4 coated paramagnetic beads (20 uL) for 30 minutes at room temperature. The cells and beads were placed next to a permanent magnet positioned such that the magnetic field was in a direction perpendicular to gravity. Bead:cell complexes were washed (3 times in RPMI 1640) and lysed in a detergent solution. Assay Reagent (luciferin/luciferase) was added to the samples ATP production was determined as described above.. FIG. 2 shows the results of this assay. An increase in ATP over background can be seen at all time points. The response is clearly evident at 4 hours of incubation, peaks at 8 hours, and remains high at 24 hrs post stimulation.

This example demonstrates that the method of the present invention can be successfully utilized to detect the response of a subset of T lymphocytes to a mitogen. The PHA used in Examples 1 and 2 was the same; Examples 1 and 2 illustrate differing incubation times.

### Example 3

### T-lymphocyte Response: Co-stimulation with Antigen and Antibody Coated Paramagnetic Particles

A blood sample was obtained from a normal donor and collected in heparin as an anticoagulant. Aliquots of the blood (50 uL) were diluted 1:2 in RPMI 1640 and placed into wells of a 96-well microtiter plate. Replicates received either no addition, or the antigen Tetanus Toxoid at 1 μg/mL final concentration plus anti-CD3/CD28 coated paramagnetic beads (0.5 uL) (Dynal, Oslo, Norway). Samples were incubated for various times (30 min, 1 hr, 2 hr, and 3 hr), at 37° C. After incubation, the 96-well strips were removed and placed in a magnet tray. Bead:cell complexes were washed (3 times in a wash buffer containing 1% BSA) and lysed in a hypotonic solution. An aliquot of the sample (50 uL) was removed to an opaque plate and Assay Reagent (luciferin/luciferase) was added. The samples were read and Relative Light Units were determined. The ATP production was then calculated from an ATP calibrator curve.

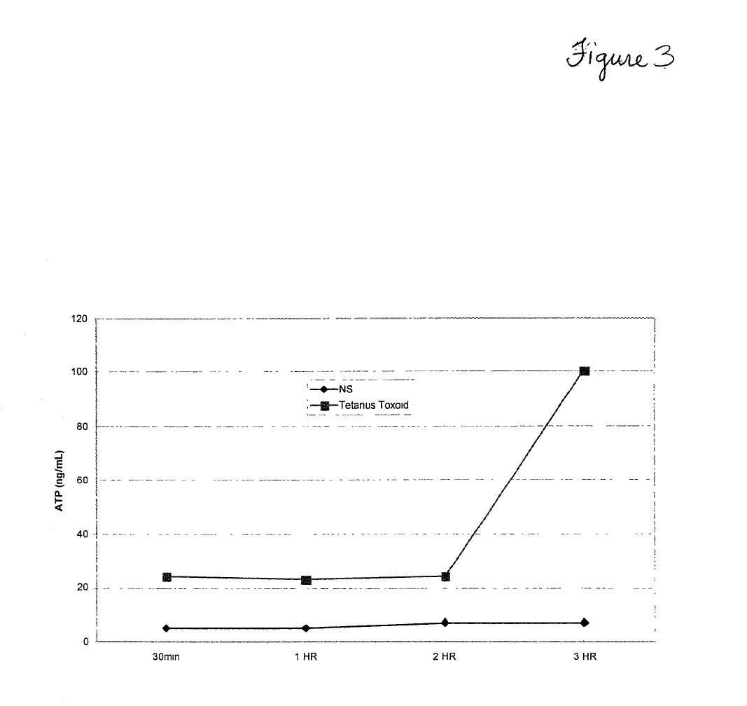

FIG. 3 shows the results of this assay. As can be seen, an increase in ATP over background is observed at the 30 minute time point and continues through the 3 hour time point.

This example demonstrates that the methods of the present invention can successfully detect T-cell activation resulting from co-incubation of the T-cells with an antigen and a costimulant at times at early as 30 minutes.

### Example 4

### T-lymphocyte Response in HIV Infected Individuals

Blood samples were obtained from a normal and HIV infected donors and collected in heparin as an anti-coagulant. Aliquots of the blood (25 uL) were diluted 1:4 in RPMI 1640 and placed into wells of a 96-well microtiter plate. Replicates received either no addition, or PHA at 10 ug/mL final concentration. Samples were incubated overnight at 37° C. After incubation, anti-CD4 coated paramagnetic beads (20 uL) were added to the samples and incubated at room temperature for 30 minutes. The 96-well strips were removed and placed in a magnet tray. Bead:cell complexes were washed (3 times in a wash buffer containing 1% Bovine Serum Albumin (BSA) (Intergen, Newark, N.J.) and lysed in a hypotonic solution. An aliquot of the sample (50 uL) was removed to an opaque plate and Assay Reagent (luciferin/luciferase) was added. The samples were read and Relative Light Units were determined. The ATP production was then calculated from an ATP calibrator curve.

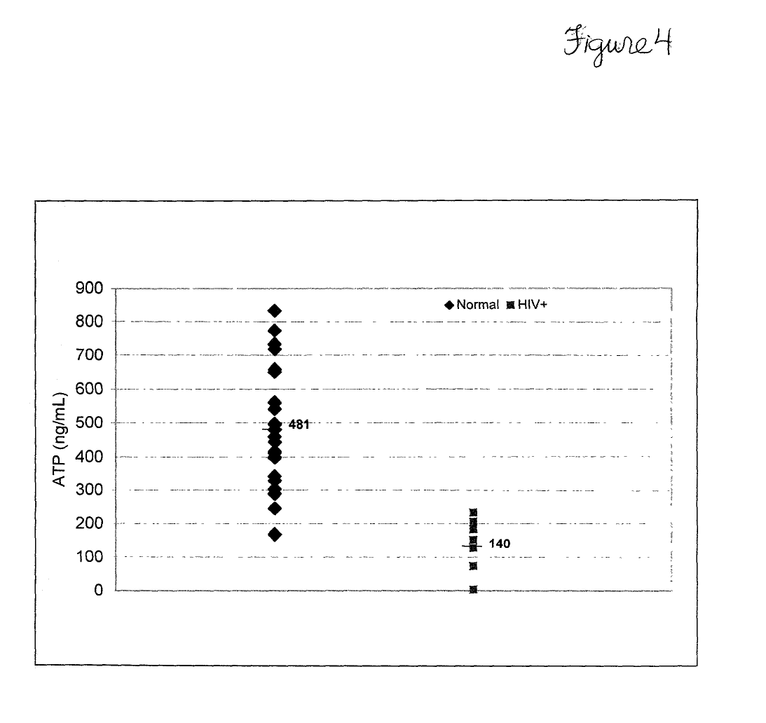

FIG. 4 shows the results of this assay. As can be seen, the ATP response to PHA of the immunosuppressed (HIV+) population is lower than the normal donor population.

This example demonstrates that the method of the present invention can be successfully utilized to detect or corroborate immunosupression in human subjects.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for detecting lymphocyte activation comprising the steps of:
incubating a sample containing a mixed population of cell types including a plurality of subsets of lymphocytes, wherein each subset includes lymphocytes with characteristic determinants that distinguish one subset from another, with at least one inducing agent selected from the group consisting of mitogens, antigens and co-stimulants;
separating a selected subset of lymphocytes from said sample;
lysing lymphocytes in said selected subset to release an intracellular component selected from the group consisting of ATP, NADP, and PCNA;
detecting a level of said intracellular component; and,
assessing lymphocyte activation for said selected subset of lymphocytes from said level of intracellular component detected in said detecting step, wherein the total time required for performing all steps is no greater than one hour.

2. The method of claim 1 wherein said at least one inducing agent comprises a co-stimulant.

3. A method for detecting lymphocyte activation comprising the steps of:
incubating a sample containing a mixed population of cell types including a plurality of subsets of lymphocytes, wherein each subset includes lymphocytes with characteristic determinants that distinguish one subset from another, with at least one inducing agent selected from the group consisting of mitogens, antigens and co-stimulants;
separating a selected subset of lymphocytes from said sample;
lysing lymphocytes in said selected subset to release an intracellular component selected from the group consisting of ATP, NADP, and PCNA;
detecting a level of said intracellular component; and,
assessing lymphocyte activation for said selected subset of lymphocytes from said level of intracellular component detected in said detecting step, wherein the total time required for performing all steps is less than three hours.

4. The method of claim 3 wherein said at least one inducing agent comprises a co-stimulant.

5. A method for detecting lymphocyte activation comprising the steps of:
incubating a sample containing a mixed population of cell types including a plurality of subsets of lymphocytes, wherein each subset includes lymphocytes with characteristic determinants that distinguish one subset from another, with at least one inducing agent selected from the group consisting of mitogens, antigens and co-stimulants;
separating a selected subset of lymphocytes from said sample;
lysing lymphocytes in said selected subset to release an intracellular component selected from the group consisting of ATP, NADP, and PCNA;
detecting a level of said intracellular component; and,
assessing lymphocyte activation for said selected subset of lymphocytes from said level of intracellular component detected in said detecting step, wherein the total time required for performing all steps is less than four hours.

6. The method of claim 5 wherein said at least one inducing agent comprises a co-stimulant.

7. A method for detecting lymphocyte activation comprising the steps of:
incubating a sample containing a mixed population of cell types including a plurality of subsets of lymphocytes, wherein each subset includes lymphocytes with characteristic determinants that distinguish one subset from another, with at least one inducing agent selected from the group consisting of mitogens, antigens and co-stimulants;
separating a selected subset of lymphocytes from said sample;
lysing lymphocytes in said selected subset to release an intracellular component selected from the group consisting of ATP, NADP, and PCNA;
detecting a level of said intracellular component; and,
assessing lymphocyte activation for said selected subset of lymphocytes from said level of intracellular component detected in said detecting step, wherein the total time required for performing all steps is less than 24 hours.

8. The method of claim 7 wherein said at least one inducing agent comprises a co-stimulant.
